(19) United States
(12) Patent Application Publication
(10) Pub. No.: US 2011/0028946 A1
(43) Pub. Date: Feb. 3, 2011

(54) Medical Instrument Surgical Display Device

(76) Inventor: Gail D. Watson, Flint, MI (US)

Correspondence Address: HARNESS, DICKEY & PIERCE, P.L.C., P.O. BOX 828, BLOOMFIELD HILLS, MI, 48303, US

(21) Appl. No.: 12/837,135

(22) Filed: Jul. 15, 2010

Related U.S. Application Data
(60) Provisional application No. 61/228,993, filed on Jul. 28, 2009.

(51) Int. Cl. A61B 17/00

(52) U.S. Cl. 606/1

(57) ABSTRACT

A re-usable medical instrument surgical display device is provided for supporting medical instruments during a surgical procedure.

### CROSS-REFERENCE TO RELATED APPLICATIONS

This application claims the benefit of U.S. Provisional Application No. 61/228,993 filed on Jul. 28, 2009. The entire disclosure of the above application is incorporated herein by reference.

### FIELD

The present disclosure relates to a medical instrument surgical display device.

### BACKGROUND

The statements in this section merely provide background information related to the present disclosure and may not constitute prior art.

Standardized operating room procedures typically require that medical instruments be grouped into standard or basic sets. The organization of the instrument sets is determined by the surgical procedure to be performed. The instruments are subject to a cleaning and sterilization process. It is common to secure medical instrument sets that include handles onto a stringer that holds the medical instruments in an open condition so that the medical instruments go through proper cleaning and sterilization processes. The basic sets of instruments that are secured to the stringer are then stored in a sterilized condition until usage. When needed, the stringers with the basic sets of instruments are removed from sterilize storage and taken to the operating room. At that point, the basic set of medical instruments is laid out on a rolled-up or folded towel, the stringer is then removed and the instruments can be organized in a fashion which permits the instruments to be handed to the surgeon, as needed, in an efficient manner. Upon completion of the surgery, the medical instruments are then re-gathered and sent to sterile processing for decontamination and reprocessing. In addition, when towels are utilized as the surface on which the medical instruments are displayed, the towels in each surgical room are discarded after the surgical procedure, thereby adding additional expense to these surgical procedures. Accordingly, it is desirable to provide a medical instrument surgical display device that can be re-used to eliminate the added expense and waste associated with the use of towels.

### SUMMARY

A medical instrument surgical display device is provided, including a riser formed from sheet metal and having a generally V-shaped cross section on which medical instruments can be supported.

The device can be cleaned by a sterilization process along with the medical instruments after the surgical procedure is completed and can be re-used repeatedly. The surgical display device eliminates the need for additional towels or other supporting structure, since the device can be utilized to support the medical instruments during the surgical procedure.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of the medical instrument surgical display device according to the principles of the present disclosure;

FIG. 2 is a top view of the medical instrument surgical display device; and

FIG. 3 is an end view of the medical instrument surgical display device according to the principles of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

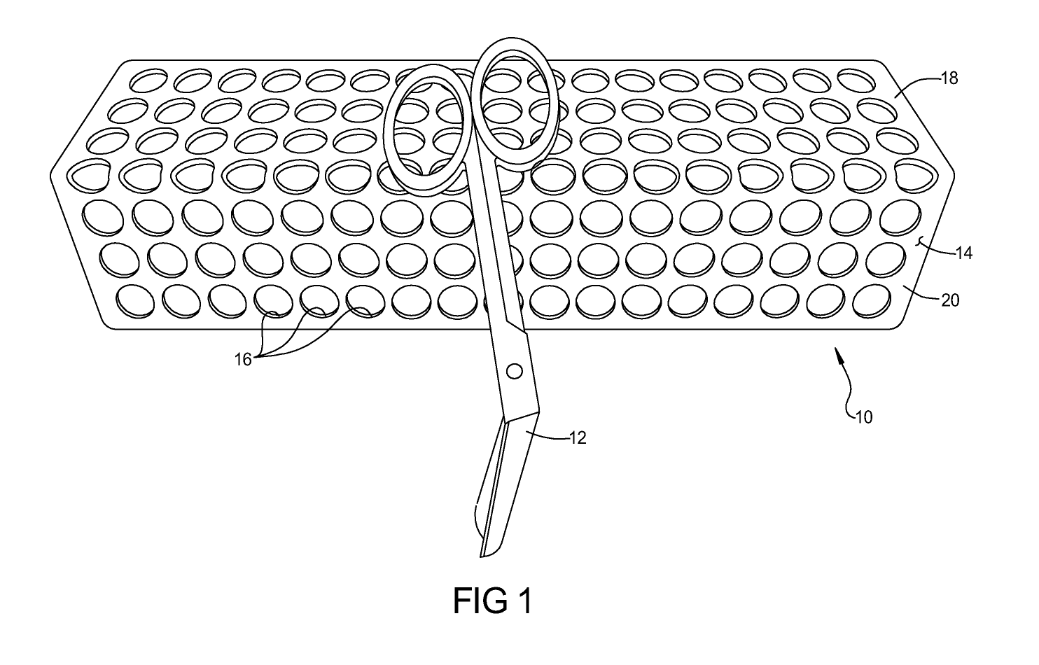

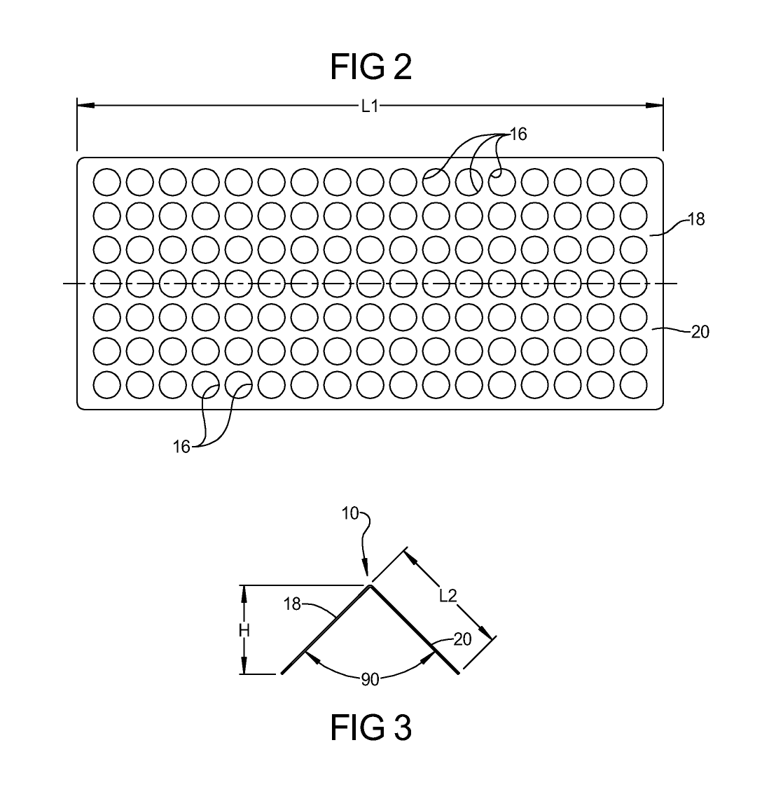

With reference to FIGS. 1-3, the medical instrument sterilization surgical display device 10 will now be described. The device 10 is employed for supporting a plurality of medical instruments during a surgical procedure. An exemplary medical instrument 12 is depicted for purposes of illustrating the operation of the device 10. The medical instrument surgical display device 10 receives a plurality of medical instruments in the manner shown to organize the instruments for usage during surgery.

Medical instrument surgical display device 10 can take the form as illustrated in FIGS. 1-3 and can include a sheet metal plate 14 formed into a generally V-shaped configuration and including a plurality of apertures 16 extending therethrough which facilitate steam penetration through the plate 14 during sterilization and decontamination of the device 10.

The device 10 can have varying lengths L1 ranging in general from approximately 4″ long to 24″ long depending upon the specific surgical procedure being performed and the number of instruments that are needed to be supported. The size, spacing, and pattern of the perforations 16 can be varied in order to provide adequate steam penetration during sterilization. The medical instrument surgical display device 10 can be utilized to support a string of medical instruments during a decontamination and/or sterilization process within a sterilization device.

According to one aspect of the present disclosure, the device 10 can be provided with a bend of approximately 90° thereby forming two legs 18, 20 to create the V-shaped configuration. Each leg has first and second end edges (18a, 18b; 20a, 20b) and upper and lower edges 18c, 20c; 18d, 20d with the upper edges 18c, 20c joined together to form an apex. The other bend angles can be utilized between 30° and 150°. The apex can include a bend radius of 0.030 inches, although other bend radii can be used. Each leg 18, 20 of the device can be between 1.5″ and 3″ in length L2 in order to provide an appropriate support height H of approximately 1″ to 2″. The perforation pattern can include holes having a diameter of between 0.25″ and 0.75″ with the spacing between centers being between 0.4″ and 0.8″. Other hole sizes and spacings can be utilized. The holes can be aligned along the apex.

It is estimated that the cost for the use of towels for supporting medical instruments can be approximately $1.75 per surgery with the towels being unable to be reused for subsequent procedures. With the re-usable medical instrument surgical display device 10 according to the principles of the present disclosure, the device can be re-sterilized along with the medical instruments and reused thereby eliminating the cost of towels from each surgical procedure, as well as reducing the waste produced. It is estimated that the one time purchase price of the surgical display device 10 can be recouped after two surgeries. Therefore, it is estimated that the elimination of towels from a surgical unit based upon the use of the surgical display device 10 could save hospitals up to $10,000 per year depending on the number of surgeries typically performed.

It should be noted that the device 10 can be made of surgical grade stainless steel or other materials that do not tarnish and which can withstand the temperatures associated with a sterilization process such as engineering plastic and other metals, either plated or un-plated.

What is claimed is:

1. A medical instrument surgical display device, comprising:
a perforated plate having a first leg and a second leg, each having first and second end edges and upper and lower edges, said upper edges of said first and second legs being directly joined along an apex and forming a generally V-shaped cross-section.

2. The medical instrument surgical display device according to claim 1, wherein said perforated plate includes a plurality of apertures.

3. The medical instrument surgical display device according to claim 2, wherein said plurality of apertures have a diameter of at least 0.25 inches.

4. The medical instrument surgical display device according to claim 3, wherein said plurality of apertures have a center-to-center spacing with adjacent apertures of not greater than 0.8 inches.

5. The medical instrument surgical display device according to claim 2, wherein said first and second legs have a bend angle therebetween of approximately 90 degrees.

6. The medical instrument surgical display device according to claim 2, wherein said apex includes a plurality of apertures therein.

7. The medical instrument surgical display device according to claim 1, wherein said perforated plate is made from stainless steel.
